(19) United States
(12) Patent Application Publication
(10) Pub. No.: US 2005/0249791 A1
(43) Pub. Date: Nov. 10, 2005

(54) Antimicrobial articles

(75) Inventors: Terry R. Hobbs, St. Paul, MN (US); John M. Sebastian, Maplewood, MN (US); Stefan H. Gryska, Woodbury, MN (US); Donald H. Lucast, North St. Paul, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/841,858

(22) Filed: May 7, 2004

(51) Int. Cl. A61F 13/00; A61K 9/70

(52) U.S. Cl. 424/443

(57) ABSTRACT

An antimicrobial article is disclosed comprising a layer of a thermoplastic polymer, and an adhesive layer having a antimicrobial agent dispersed therein. The antimicrobial article is useful, for example, surgical tapes, surgical drapes and wound dressings, and as disposable surfaces for food preparation and handling.

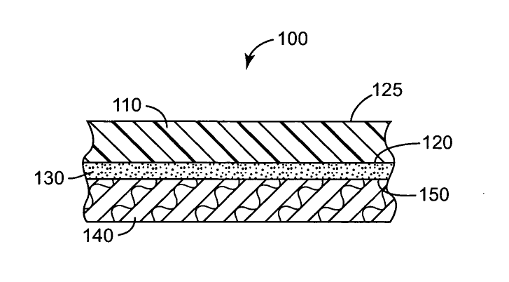

### FIELD OF THE INVENTION

The present invention relates to an antimicrobial article comprising a layer of a thermoplastic polymer, and an adhesive layer having an antimicrobial agent dispersed therein. The present invention also relates to a method of making such articles.

### BACKGROUND OF THE INVENTION

The control of mold, mildew, algae, fungi, and other microbes or microorganisms in moist or humid environments has long been a matter of concern. Antimicrobials such as mildewcides, antiseptics, disinfectants, sanitizers, germicides, algaecides, slimicides, antifouling agents, or preservatives are typically employed to remove microbes from an area and prevent their recurrence.

Antimicrobial articles have been prepared by incorporation of antimicrobial agents directly into a polymeric hot melt prior to extrusion. This method allows the antimicrobial agents to be directly incorporated into the thermoplastic polymer. Melt processing, however, requires very high temperatures, e.g., 300° C. or higher. At such temperatures, many antimicrobial agents, especially organic molecules, face problems with thermal and oxidative stability and volatility. Further, the antimicrobial activity of such articles may be compromised by wear and exposure, and the antimicrobial agent may be difficult to replenish without replacing the article. Thus, alternative methods for the preparation of antimicrobial articles are needed.

### SUMMARY

Accordingly, there is a need for thermoplastic polymer articles with an antimicrobial surface. As will be set forth in detail below, the present invention solves this problem by dispersing an antimicrobial agent in an adhesive layer bonded, adhered or otherwise affixed to the thermoplastic polymer layer of the article. The antimicrobial agent may be any known in the art, that may be compounded with an adhesive, and that will migrate from the adhesive layer to render the thermoplastic polymer layer (or surface thereof) antimicrobial. The polymer layer may be in the form of a nonporous film, a porous film, a foam, a membrane or a fibrous layer, such as a woven or nonwoven fabric.

The present invention provides an antimicrobial article comprising a polymeric layer having a first antimicrobial surface and a second surface having an adhesive layer bonded, laminated, adhered, or otherwise affixed thereto; said adhesive layer comprising sufficient antimicrobial agent dispersed therein which migrates to said first surface of said polymeric layer, rendering the first surface of said polymeric layer antimicrobial. The antimicrobial articles are useful, for example, as surgical tapes, surgical drapes and wound dressings, and as disposable surfaces for food preparation and handling.

While a range of antimicrobial agent concentrations may be used in the practice of the invention, generally the adhesive layer will contain at least 0.25 wt. % of at least one antimicrobial agent, based on the total weight of the adhesive layer. Preferably the pressure sensitive adhesive layer comprises from at least 0.25 percent by weight up to and including 40 percent by weight of at least one antimicrobial agent, based on the total weight of the adhesive layer.

It will be understood that in connection with the present invention the use of the term “dispersed therein” denotes merely the initial presence of the antimicrobial agent in the adhesive layer without limitation as to where the antimicrobial agent may subsequently migrate. Thus the antimicrobial agent may be initially uniformly dispersed in the bulk of the adhesive or may have migrated to the surface of the thermoplastic polymer layer.

As used herein, “antimicrobial”, with reference to the article, is used only to refer to the surface characteristics of the thermoplastic polymer layer: that the surface kills or suppresses the growth of microorganisms. As used herein “antimicrobial agent” refers to a chemical agent that kills or suppresses the growth of microorganisms, and includes, for example, germicides, bactericides, fungicides, virucides, biocides, bacteriostats, fungistats, antibiotics, and algaecides.

The present invention solves the problem of the art by providing a reservoir for antimicrobial agents in an adhesive layer adhered to thermoplastic polymer layer, in order that the surface(s) of the polymer layer is rendered antimicrobial via migration of such antimicrobial agents from the adhesive into the polymer layer, and to provide for replenishment of the antimicrobial agent, which may be lost, degraded or otherwise rendered ineffective through use or exposure.

One aspect of the present invention is a method for providing an antimicrobial article comprising a thermoplastic polymer layer and an adhesive layer, comprising the steps of: (a) dispersing into an adhesive layer at least one antimicrobial agent that provides an antimicrobial surface to the polymer layer; and (b) adhering the adhesive layer to a thermoplastic polymer layer such that the adhesive layer provides an antimicrobial agent reservoir for the polymer layer. Alternatively the method may comprise providing a thermoplastic polymer layer, and coating the same with an adhesive layer containing at least one antimicrobial agent. The adhesive layer comprises from greater than 0.25 percent by weight up to and including 40 percent by weight of at least one antimicrobial agent based on the total weight of the adhesive. A feature of the present invention is the ability to provide a reservoir of antimicrobial agent in an adhesive contacting the polymer layer to provide antimicrobial activity over a period of time.

Preferably, the compositions of the present invention include one or more surfactants, which can be nonionic, anionic, or amphoteric. It has been found that the addition of surfactants may enhance the migration and/or the efficacy of the antimicrobial agent. In certain embodiments, preferred surfactants are anionic or amphoteric surfactants selected from the group consisting of sulfonates, sulfates, phosphates, phosphonates, and ammonium sulfonate amphoterics, and mixtures thereof. In certain other embodiments, a preferred surfactant is an amine oxide or an ethoxylated derivative such as Steareth 10. Mixtures of surfactants can be used if desired.

Unexpectedly, the method of the present invention not only provides a antimicrobial surface to a polymer layer adjoining the adhesive, but also, when the reservoir adhesive adjoins a multilayer article, other layers in a composite article. More specifically, the antimicrobial agents migrate through the adjacent polymer layer into additional layers in a multilayer article. Significantly, the antimicrobial agents in a reservoir may migrate across two different layers of two different materials to render a third layer antimicrobial. Thus, another advantage of the present invention is the ability to use multilayer films that might not contain any antimicrobial agents yet are provided an antimicrobial surface via antimicrobial agents that have migrated from an adhesive layer, through intermediate layers.

Another aspect of the present invention is a thermoplastic polymer layer that is rendered antimicrobial by an adjoining adhesive delivery system for antimicrobial agents that provides an antimicrobial surface to the adjoining thermoplastic polymer layer, and wherein the thermoplastic polymer layer itself is initially not antimicrobial, prior to antimicrobial agent migration.

“Adhesive delivery system” means the use of adhesive to provide a reservoir for antimicrobial agents and to facilitate the migration of such antimicrobial agents from the adhesive layer into adjoining thermoplastic polymer layer(s), and may further provide renewal or replenishment of the antimicrobial agent during use. Use of this adhesive delivery system eliminates problems that occur in the two most common methods used for providing an antimicrobial surface to into thermoplastic polymers: extrusion and coating.

Antimicrobial agents frequently cannot be directly compounded and extruded as a melt because of the low decomposition temperatures of the antimicrobial agents. In other cases, the antimicrobial agents may interfere with polymer nucleation, or may degrade the physical properties of the thermoplastic polymer during processing. Yet further, agents that are directly compounded into thermoplastic polymers cannot be renewed.

Coating methods to provide an antimicrobial surface also have some limitations. First, the extra step required in coating a film is expensive, time consuming and involves safety and environmental issues. Many of the solvents used for coating are flammable liquids or have exposure limits that require special production facilities. Furthermore the quantity of antimicrobial agent is limited by the solubility in the coating solvent and the thickness of the coating. Yet further, such antimicrobial coatings may be abraded or otherwise removed during exposure or use. The “adhesive delivery system” of the present invention solves these problems.

The antimicrobial articles of the present invention are suitable for many purposes: including surgical tapes, dressing and drapes, wound dressings, and disposable surfaces for food preparation and handling.

### BRIEF DESCRIPTION OF THE FIGURE

The FIGURE is an exemplary cross-sectional side view of an antimicrobial article according to the present invention.

### DETAILED DESCRIPTION

Referring now to the FIGURE, exemplary antimicrobial article 100 comprises thermoplastic polymer layer 110 having major surfaces 120 and 125. Pressure sensitive adhesive layer 130 contacts major surface 120. Pressure sensitive adhesive layer 130 comprises at least one pressure sensitive adhesive and at least one antimicrobial agent. In some embodiments of the present invention, antimicrobial article 100 may further comprise a release liner 140 releasably affixed to major surface 150 of adhesive layer 130.

Without wishing to be bound by theory, it is believed that antimicrobial agent in the adhesive layer gradually migrates from the pressure sensitive adhesive layer into the thermoplastic polymer layer. During use, exposure or storage, antimicrobial agent that has diffused to the thermoplastic polymer layer may be depleted. By providing a gradual release of antimicrobial agent from the adhesive reservoir, the thermoplastic polymer layer may be provided with a continuous supply of antimicrobial agent.

It is believed that the migration of the antimicrobial agent from the adhesive layer through the thermoplastic polymer layer is a diffusion process, and therefore the T_{g} of the adhesive layer and thermoplastic polymer layers are preferably at or below 25° C., and is more preferably below about 0° C. Polymers in the glassy state are generally less permeable than those in the rubbery state, so polymers in the rubbery state are particularly useful. Heating the article may enhance the migration of the antimicrobial agent. It will be understood that the permeability of the thermoplastic polymer layer will be sufficient to deliver the desired level of antimicrobial activity during use, and is dependent on the particular antimicrobial chosen, the morphology of the thermoplastic polymer (e.g. whether fibrous, film, etc) and the end-use conditions.

If it is assumed that Fick's Second Law applies, such that there is an effective diffusion coefficient (D) that is not concentration dependent, then for 1 dimensional diffusion of a species into a semi-infinite medium, the solution of
∂C/∂t=D(∂²C/∂x²) [Fick's 2^{nd} law]
where C=C₀, x=0, t>0 [boundary condition]
and C=0, x>0, t=0 [initial condition] is found to be
C=C₀(ERFC[x/(4Dt)^{1/2}]),
   where C is the concentration of the diffusing species, t is time, x is the coordinate of the diffusion direction, and ERFC is the complementary error function. Reference may be made to The Mathematics of Diffusion, 2^{nd} Edition, J. Crank, Clarendon Press, Oxford, 1975.

Preferably, the Fick's diffusion constant, D (which is dependent on the antimicrobial agent, the polymer and temperature) is greater than 0.1×10⁻¹⁰ cm²/s, preferentially greater than 10×10⁻¹⁰ cm²/s and most preferentially greater than 100×10⁻¹⁰ cm²/s at 25° C. It is expected that films having diffusion constants in this range would experience rates of diffusion such that the concentration of the antimicrobial reaches a level about equal to half of its initial value in the adhesive (i.e. C=C₀/2 from above) within a few days. For liquid antimicrobial agents, it may be preferred for the concentration to be above the solubility limit in the adhesive. Above this limit the diffusion will be enhanced.

Examples of thermoplastic polymers for use in the thermoplastic polymer layer include polyesters, polyurethanes, polyamides and polyolefins. Preferred thermoplastic polymers are poly(alpha)olefins. Poly(alpha)olefins can include the normally solid, homo-, co- and terpolymers of aliphatic mono- 1-olefins (alpha olefins) as they are generally recognized in the art. Usually, the monomers employed in making such poly(alpha)olefins contain about 2 to 10 carbon atoms per molecule, though higher molecular weight monomers sometimes are used as comonomers. The invention is applicable also to blends of the polymers and copolymers prepared mechanically or in situ. Examples of useful monomers that can be employed to prepare the thermoplastic polymers include ethylene, propylene, butene, pentene, 4-methyl-pentene, hexene, and octene, alone, or in admixture, or in sequential polymerization systems. Examples of preferred thermoplastic polymers include polyethylene, polypropylene, propylene/ethylene copolymers, polybutylene, polyurethanes and blends thereof. Processes for preparing the thermoplastic polymers are well known, and the invention is not limited to a polymer made with a particular process.

The thermoplastic polymer layer may be in the form of a film, foam, membrane or fibrous layer and may be oriented or unoriented. As used herein, the terms “fiber” and “fibrous” refer to particulate matter, generally thermoplastic resin, wherein the length to diameter ratio of the particulate matter is greater than or equal to about 10. Fiber diameters may range from about 0.5 micrometers up to at least 1,000 micrometers. Each fiber may have a variety of cross-sectional geometries, may be solid or hollow, and may be colored by, e.g., incorporating dye or pigment into the polymer melt prior to extrusion. For purposes of this invention, a “film” is distinguished from a “membrane” in that any porosity present in a film does not transcend the entire thickness of the film, whereas at least some porosity present in a membrane does transcend the entire thickness of the membrane to provide a fluid conduit between opposing surfaces.

Useful fibrous thermoplastic polymer layers include woven, knitted, and nonwoven fabrics. The thermoplastic polymer layer may have any thickness, but typically, the thickness is in a range of from at least 10, 25, or 1000 micrometers up to and including 0.5, 2.5, or even 5 millimeters or more. The thermoplastic polymer layer may be a single layer, or may comprise multiple layers of the same of different thermoplastic polymers. In one embodiment, the antimicrobial article may have a construction such as P¹P² . . . P¹⁰⁴ A, where P¹, P², to P^{ψ} represent thermoplastic polymer layers, and A represents an adhesive layer, having an antimicrobial agent dispersed therein. Multilayer films can be made using a variety of equipment and a number of melt-processing techniques (typically, extrusion techniques) well known in the art. Such equipment and techniques are disclosed, for example, in U.S. Pat. No. 3,565,985 (Schrenk et al.), U.S. Pat. No. 5,427,842 (Bland et al.), U.S. Pat. No. 5,589,122 (Leonard et al.), U.S. Pat. No. 5,599,602 (Leonard et al.), and U.S. Pat. No. 5,660,922 (Herridge et al.).

The fibrous thermoplastic polymer layer may include non-woven webs manufactured by any of the commonly known processes for producing nonwoven webs. For example, the fibrous nonwoven web can be made by carded, air laid, spunlaced, spunbonded or melt-blown techniques or combinations thereof. Spunbonded fibers are typically small diameter fibers that are formed by extruding molten thermoplastic polymer as filaments from a plurality of fine, usually circular capillaries of a spinneret with the diameter of the extruded fibers being rapidly reduced. Meltblown fibers are typically formed by extruding the molten thermoplastic material through a plurality of fine, usually circular, die capillaries as molten threads or filaments into a high velocity, usually heated gas (e.g. air) stream which attenuates the filaments of molten thermoplastic material to reduce their diameter. Thereafter, the meltblown fibers are carried by the high velocity gas stream and are deposited on a collecting surface to from a web of randomly disbursed meltblown fibers. Any of the non-woven webs may be made from a single type of fiber or two or more fibers that differ in the type of thermoplastic polymer and/or thickness.

Further details on the manufacturing method of non-woven webs of this invention may be found in Wente, Superfine Thermoplastic Fibers, 48 INDUS. ENG. CHEM. 1342(1956), or in Wente et al., Manufacture Of Superfine Organic Fibers, (Naval Research Laboratories Report No. 4364, 1954).

Where the polymer layer is a microporous membrane, the membrane has a structure that enables fluids to flow through it. Nonetheless, it is believed that the antimicrobial agent migrates through the bulk of the polymeric matrix, rather than through the pores. The effective pore size is at least several times the mean free path of the flowing molecules, namely from several micrometers down to about 100 Angstroms. Such sheets are generally opaque, even when made of transparent material, because the surfaces and the internal structure scatter visible light.

There are several methods known in the art to prepare microporous membranes. A preferred method for producing the microporous membranes of the present invention utilizes the phase separation phenomenon that utilizes either liquid-liquid or solid-liquid phase separation. The method for producing microporous structures using these techniques usually involves melt blending the polymer with a compatible liquid that is miscible with the polymer at the casting or extrusion temperature, forming a shaped article of the melt blend, and cooling the shaped article to a temperature at which the polymer phase separates from the compatible liquid. Microporosity can be imparted to the resultant structure by, for example, (i) orienting the structure in at least one direction; (ii) removing the compatible liquid and then orienting the structure in at least one direction; or (iii) orienting the structure in at least one direction and then removing the compatible liquid. The cooling step for films is usually accomplished by contacting the film with a chill roll. This results in a thin skin being formed on the side of the membranes that contacts the chill roll.

Such methods are described, for example, in U.S. Pat. No. 4,247,498 (Castro), U.S. Pat. No. 4,539,256 (Shipman), U.S. Pat. No. 4,726,989 (Mrozinski) and U.S. Pat. No. 4,867,881 (Kinzer). Particulate-filled microporous membranes such as those described in, for example, U.S. Pat. No. 4,777,073 (Sheth), U.S. Pat. No. 4,861,644 (Young et al.), and U.S. Pat. No. 5,176,953 (Jacoby et al. ), as well as JP 61-264031 (Mitsubishi Kasei K K), can also be utilized. Microporosity can be imparted to such particulate-filled films by, for example, orienting the film in at least one direction.

The thermoplastic polymer layer, whether film, membrane or fibrous, may comprise a pattern of elevated areas or relatively thick portions, separated by valleys, or relatively thin portions. The elevated areas take the form of ridges, mounds, peaks, cylinders, grooves or other embossments which may be uniform or varied in shape and dimensions and are generally disposed in a regular arrangement or pattern. “Pattern” does not necessarily refer to a regular repeating array, but may mean a random array of features having the same or different sizes. Patterns suitable for the practice of this invention include four-sided square pyramids, truncated four-sided square pyramids, cones, straight lines, wavy lines, square or rectangular blocks, hemispheres, grooves and the like and are imparted to at least a portion of the thermoplastic polymer layer. An individual feature of the pattern is referred to as an embossment. The number and spacing of embossments, as well as the nature of the individual embossment, such as its depth, degree of sharp reflecting edges, and shape can be varied as well. The terms “pattern” and “embossment” are used without reference to the process of application.

A plurality of embossments may be formed on the thermoplastic polymer layer. There are typically about 5 to 20 embossments per lineal centimeter. The embossments can be of any suitable depth, as long as the mechanical properties of the films are sufficient for the desired end use after the embossments have been formed. The depth of an embossment typically ranges from 10 to about 90 percent of the thickness of the oriented thermoplastic film. Preferably, the depth of an embossment typically ranges from 25 to 75 percent of the thickness of the thermoplastic polymer.

Embossing refers to a process in which a pattern is impressed into the surface of an article. Embossing is typically accomplished by means of a male pattern formed on a hard material such as a metal layer on an embossing roll. Those skilled in the art recognize that embossing can be done by several methods, including the use of a continuous tooled belt or sleeve. Preferred metal layers include those comprising nickel, copper, steel, and stainless steel. Patterns are typically acid etched or machined into the metal layer and can have a wide variety of sizes and shapes. Any pattern that can be scribed into a metal surface can be used in the practice of this invention. One useful embossing method is described in Assignee's U.S. Pat. No. 6,514,597, (Strobel et al.), incorporated herein by reference.

Embossing can be carried out by any means known in the art. The preferred method of embossing is to move the softened thermoplastic polymer layer (prior to coating with the adhesive layer) through a nip having an embossing surface. “Nip” refers to two rolls in proximity that apply pressure on a film when the film passes between them. The embossing surface contacts the film with sufficient force to create embossments in the softened surface of the thermoplastic polymer layer. The embossed surface is then cooled by any of a number of methods to reduce the temperature of the softened surface to below its softening temperature before the article has experienced a significant change in bulk properties resulting from prior orientation. Such methods include moving the film over one or more chilled rollers, delivering it to a water bath, or cooling by air or other gases, such as by use of an air knife.

Useful antimicrobial agents typically include any agent that kills or suppresses the growth of microorganisms such as germicides, bactericides, fungicides, virucides, biocides, bacteriostats, fungistats, antibiotics, and algicides. The antimicrobials may be selected from those that are nonreactive with the adhesive or thermoplastic polymer layer, and can migrate from the adhesive layer to the thermoplastic polymer layer to render it antimicrobial. Antimicrobial agents may be selected based on the type of microorganisms the antimicrobial article will encounter in a particular use.

Examples of antimicrobials may include chemical agents of selective toxicity, i.e. injurious to one kind of organism, but not harmful to another. Antimicrobial agents of low selectivity (injurious to all organisms) may include antimicrobial acids, esters, alcohols, peroxides, aldehydes (and aldehyde-releasing compounds), halogens (and halogen-releasing compounds), phenols, cresols, quaternary ammonium compounds, bleach and biguanides.

Antimicrobial agents of moderate selectivity include antibiotics such as bacitracin and polymyxin; acridine and triphenyl methane dyes; organic arsenic compounds, organic mercury compounds, and silver compounds.

Antimicrobial agents of high selectivity include synthetic antibacterial agents such as p-aminosalicylic acid, isonicotinic acid, sulfonamides, trimethoprim, metronidazole, and 4-quinolone derivatives; synthetic antifungal agents such as imidazole derivatives such as clotrimazole, synthetic antiviral agents such as amantadine, idoxuridine, cytarabine, acyclovir, and zidovudine; antibacterial antibiotics such as aminoglycoside-aminocyclitol, beta lactamase inhibitors, lincomycoins, macrolides, rifamycins and tetracyclins; and antifungal antibiotics such as griseofulvin, amphotericin, systatin and imidazoles.

Preferred examples of antimicrobial agents include iodine and its complexed forms, which are commonly referred to as iodophors. lodophors are iodine complexes with polyethylene glycol and its derivatives, N-vinyl caprolactam containing polymers such as polyvinylpyrrolidone, as well as other polymers or polar molecules that tend to hydrogen bond with hydrogen iodide or hydrogen triiodide or complex with salts such as sodium or potassium triiodide. A particularly preferred iodophor is povidone-iodine and most preferably povidone-iodine USP. Other preferred antimicrobials include chlorhexidine salts such as chlorhexidine gluconate (CHG); parachlorometaxylenol (PCMX); triclosan; hexachlorophene; fatty acid monoesters of glycerin and propylene glycol such as glycerol monolaurate, glycerol monocaprylate, glycerol monocaprate, propylene glycol monolaurate, propylene glycol monocaprylate, propylene glycol monocaprate; phenols; polymers that include a C₁₂-C₂₂ hydrophobe and a quaternary ammonium group; polyquatemary amines such as polyhexamethylene biguanide; quaternary silanes; hydrogen peroxide; silver and silver salts such as silver chloride, silver oxide and silver sulfadiazine; and the like. The most preferred antimicrobial agent is triclosan since it is capable of ensuring long-term antimicrobial efficacy at relatively low concentrations and does not promote antimicrobial resistance.

Further reference may be made to Seymour S. Block, Disinfection , Sterilization and Preservation, 4^{th} Edition, Lea & Febiger, Philadelphia, Pa., 1991. The specific antimicrobial may be selected based on the desired application substrate (e.g. human contact), and the specific organism to be killed or suppressed. Various combinations of antimicrobial agents can be used in the present invention.

Any adhesive suitable for use with thermoplastic polymers, that can also serve as a reservoir for antimicrobial agents, and that is non-reactive toward the antimicrobial agents, can be used in the present invention. Adhesives can include hot melt adhesives, actinic radiation reactive adhesives, and the like. The adhesives can be solvent-based adhesives, 100% solids adhesives, or latex-based adhesives. Reference may be made to Handbook of Pressure Sensitive Adhesive Technology, Second Edition, D. Satas, Editor, Van Nostrand, Rheinhold, 1989. Preferably the adhesive is a pressure sensitive adhesive. “Pressure sensitive adhesive” means an adhesive that is aggressively and permanently tacky at room temperature and firmly adheres to a variety of dissimilar surfaces upon mere contact without the need of more than finger or hand pressure, and has a sufficiently cohesive holding and elastic nature so that they can be handled with the fingers and removed from smooth surfaces without leaving a residue.

Suitable pressure sensitive adhesives include, for example, those based on natural rubbers, synthetic rubbers, styrene block copolymers, polyvinyl ethers, poly (meth)acrylates (including both acrylates and methacrylates), polyurethanes, polyureas, polyolefins, and silicones. The pressure sensitive adhesive may comprise an inherently tacky material, or if desired, tackifiers may be added to a tacky or non-tacky base material to form the pressure sensitive adhesive. Useful tackifiers include, for example, rosin ester resins, aromatic hydrocarbon resins, aliphatic hydrocarbon resins, and terpene resins. Other materials can be added for special purposes, including, for example, plasticizers, hydrogenated butyl rubber, glass beads, conductive particles, filler, dyes, pigments, and combinations thereof.

Pressure sensitive adhesives are commercially available from a number of sources including, for example, 3M Company, Saint Paul, Minn. Further examples of useful pressure sensitive adhesives include those generally described in U.S. Pat. No. 4,112,213 (Waldman); U.S. Pat. No. 4,917,928 (Heinecke); U.S. Pat. No. 4,917,929 (Heinecke); U.S. Pat. No. 5,141,790 (Calhoun); U.S. Pat. No. 5,045,386 (Stan et al.); U.S. Pat. No. 5,229,207 (Paquette et al.); U.S. Pat. No. 5,296,277 (Wilson et al.); U.S. Pat. No. 5,670,557 (Dietz et al.); and U.S. Pat. No. 6,232,366 (Wang et al.); the disclosures of which as incorporated herein by reference.

The pressure sensitive adhesive layer may have any thickness. For example, the pressure sensitive adhesive layer may have a thickness in a range of from at least 25, 100, or 250 micrometers up to and including 500, 1000, or 2500 micrometers or even more.

Depending on the specific thermoplastic polymer layer chosen and intended application, the pressure sensitive adhesive layer may be selected such that it cannot be mechanically separated from the thermoplastic polymer layer without damaging the thermoplastic polymer layer. This may be desirable, for example, in the case that two thermoplastic polymer layers are bonded together by the pressure sensitive adhesive layer.

The pressure sensitive adhesive layer may be continuous, for example, as a continuous adhesive film on one major surface of the thermoplastic polymer layer. Alternatively, the pressure sensitive adhesive layer can be a discontinuous layer. In one embodiment, the pressure sensitive adhesive layer may have the shape of an alphanumeric character or graphic image. In another embodiment, the adhesive may be on one or more edges, or the periphery of the thermoplastic polymer layer. Suitable methods for applying the pressure sensitive adhesive layer include, for example, roll coating, gravure coating, curtain coating, spray coating, screen printing, with the method typically chosen based on the type of coating desired.

In one embodiment, the antimicrobial article may further comprise a release liner, for example, to protect the adhesive before usage. Examples of release liners include silicone coated kraft paper, silicone coated polyethylene coated paper, silicone coated or non-coated polymeric materials such as polyethylene or polypropylene, as well as the aforementioned base materials coated with polymeric release agents such as silicone urea, urethanes, and long chain alkyl acrylates, such as generally described in U.S. Pat. No. 3,997,702 (Schurb et al.); U.S. Pat. No. 4,313,988 (Koshar et al.); U.S. Pat. No. 4,614,667 (Larson et al.); U.S. Pat. No. 5,202,190 (Kantner et al.); and U.S. Pat. No. 5,290,615 (Tushaus et al.); the disclosures of which are incorporated by reference herein. Suitable commercially available release liners include those available under the trade designation “POLYSLIK” from Rexam Release of Oakbrook, Ill., and under the trade designation “EXHERE” from P. H. Glatfelter Company of Spring Grove, Pa.

It is particularly desirable when formulating with an adhesive to include one or more surfactants to enhance migration of the antimicrobial agent and/or increase the antimicrobial activity. If used, one or more surfactants are generally added to the adhesive layer of the antimicrobial article in an amount of at least about 0.05 wt-%, based on the total weight of the adhesive. Preferably, one or more surfactants are generally added in an amount of no greater than about 30 wt-%, more preferably no greater than about 20 wt-%, even more preferably no greater than about 10 wt-%, and most preferably no greater than about 5 wt-%, based on the total weight of the adhesive. Useful classes of surfactants include nonionic, anionic, and amphoteric surfactants.

One useful class of nonionic surfactants include the condensation products of a higher aliphatic alcohol, such as a fatty alcohol, containing about 8 to about 20 carbon atoms, in a straight or branched chain configuration, condensed with about 3 to about 100 moles, preferably about 5 to about 40 moles, most preferably about 5 to about 20 moles of ethylene oxide. Examples of such nonionic ethoxylated fatty alcohol surfactants are the Tergitol™ 15- S series from Union Carbide and Brij™ surfactants from ICI. Tergitol™ 15-S Surfactants include C₁₁-C₁₅ secondary alcohol polyethyleneglycol ethers. Brij™ 97 surfactant is polyoxyethylene(10) oleyl ether; Brij™ 58 surfactant is polyoxyethylene(20) cetyl ether; and Brij™ 76 surfactant is polyoxyethylene(10) stearyl ether.

Another useful class of nonionic surfactants include the polyethylene oxide condensates of one mole of alkyl phenol containing from about 6 to 12 carbon atoms in a straight or branched chain configuration, with about 3 to about 100 moles, preferably about 5 to about 40 moles, most preferably about 5 to about 20 moles of ethylene oxide to achieve the above defined HLB. Examples of nonreactive nonionic surfactants are the Igepal™ CO and CA series from Rhone-Poulenc. Igepal™ CO surfactants include nonylphenoxy poly(ethyleneoxy) ethanols. Igepal™ CA surfactants include octylphenoxy poly(ethyleneoxy) ethanols.

Another useful class of nonionic surfactants include block copolymers of ethylene oxide and propylene oxide or butylene oxide with HLB values of about 6 to about 19, preferably about 9 to about 18, and most preferably about 10 to about 16. Examples of such nonionic block copolymer surfactants (known as poloxamers) are the Pluronic™ and Tetronic™ series of surfactants from BASF. Pluronic™ surfactants include ethylene oxide-propylene oxide block copolymers. Tetronic™ surfactants include ethylene oxide-propylene oxide block copolymers. A preferred example is Polaxamer 124 or Pluronic L44, which are liquids at room temperature and have HLB values of 12 to 18.

Still other useful nonionic surfactants include sorbitan fatty acid esters, polyoxyethylene sorbitan fatty acid esters and polyoxyethylene stearates having HLBs of about 6 to about 19, preferably about 9 to about 18, and most preferably about 10 to about 16. Examples of such fatty acid ester nonionic surfactants are the Span™, Tween™, and Myrj™ surfactants from ICI (now Uniqema). Span™ surfactants include C₁₂-C₁₈ sorbitan monoesters. Tween™ surfactants include poly(ethylene oxide) C₁₂-C₁₈ sorbitan monoesters. Myrj™ surfactants include poly(ethylene oxide) stearates.

Particularly suitable hydrocarbon nonionic surfactants include polyoxyethylene alkyl ethers, polyoxyethylene alkyl-phenyl ethers, polyoxyethylene acyl esters, sorbitan fatty acid esters, polyoxyethylene alkylamines, polyoxyethylene alkylamides, polyoxyethylene lauryl ether, polyoxyethylene cetyl ether, polyoxyethylene stearyl ether, polyoxyethylene oleyl ether, polyoxyethylene octylphenyl ether, polyoxyethylene nonylphenyl ether, polyethylene glycol laurate, polyethylene glycol stearate, polyethylene glycol distearate, polyethylene glycol oleate, oxyethylene-oxypropylene block copolymer, sorbitan laurate, sorbitan stearate, sorbitan distearate, sorbitan oleate, sorbitan sesquioleate, sorbitan trioleate, polyoxyethylene sorbitan laurate, polyoxyethylene sorbitan stearate, polyoxyethylene sorbitan oleate, polyoxyethylene laurylamine, polyoxyethylene laurylamide, laurylamine acetate, hard beef tallow propylenediamine dioleate, ethoxylated tetramethyldecynediol, fluoroaliphatic polymeric ester, polyether-polysiloxane copolymer, and the like.

The nonionic surfactant may correspond to the following formula: Rₕ¹-Y¹-W-Y²-Rₕ², (I) wherein:
W represents a polyoxyalkylene group, preferably a polyoxyethylene group; Y¹ and y² independently represent an oxygen or sulfur atom or a group of the formula —CO—, —COO—, —NH—, —CONH—, or —N(R)—, where R is an alkyl group or an aryl group;
Rₕ¹ represents an alkyl or an aryl group, or a combination thereof, that may be substituted or unsubstituted and that contains from 2 to about 20 carbon atoms whose skeletal chain may be straight-chained, branched, or, if sufficiently large, cyclic, or any combination thereof, the skeletal chain can also optionally include one or more catenary heteroatoms (such as oxygen, hexavalent sulfur, and trivalent nitrogen atoms) bonded to the carbon atoms of the skeletal chain, and
Rₕ² represents a hydrogen atom or is an alkyl or an aryl group, or a combination thereof, that may be substituted or unsubstituted and that contains from 2 to about 20 carbon atoms whose skeletal chain may be straight-chained, branched, or, if sufficiently large, cyclic, or any combination thereof, the skeletal chain can also optionally include one or more catenary heteroatoms such as oxygen, hexavalent sulfur, and trivalent nitrogen atoms bonded to the carbon atoms of the skeletal chain.

One or both of the depicted Rₕ¹ and Rₕ² may contain a polydialkylsiloxane group of the formula: 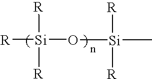
where all the depicted R groups are independently selected as alkyl or aryl groups having from 1 to about 10 carbon atoms that may be substituted or unsubstituted, straight-chained or branched, cyclic or acyclic, and may contain one or more catenary heteroatoms;

The variable W in the hydrocarbon surfactants according to the above Formula I is a polyoxyalkylene group (OR¹)s, where R¹ is an alkylene group having from 2 to about 4 carbon atoms, such as —CH₂CH₂—, —CH₂CH₂CH₂—, —CH(CH₃)CH₂—, and —CH(CH₃)CH(CH₃)—, and s is a number such that the weight percent of oxyalkylene units in the hydrocarbon surfactant is between 20 and 80 percent and more preferably between 40 and 70 weight percent. The oxyalkylene units in the poly(oxyalkylene) group can be the same, such as in poly(oxypropylene) or poly(oxyethylene), or present as a mixture, such as in a hetero straight or branched chain of randomly distributed oxyethylene and oxypropylene units i.e., poly(oxyethylene-co-oxypropylene), or as in a straight or branched chain blocks of oxypropylene units.

Representative surfactants according to Formula I above include ethoxylated alkylphenols (such as the TRITON™ TX, IGEPAL™ CA and IGEPAL™ CO series, commercially available from Union Carbide Corp. and Rhone-Poulenc Corp. respectively), ethoxylated dialkylphenols (such as the IGEPAL™ DM series, also commercially available from Rhone-Poulenc Corp.), ethoxylated fatty alcohols (such as the TERGITOL™ series, commercially available from Union Carbide Corp.) and polyoxyethylene fatty acid mono- esters and diesters (such as the MAPEG™ MO and MAPEG™ DO series, commercially available from PPG Industries, Inc.).

Another class of nonionic polyoxyethylene-containing surfactants in accordance with the invention may be described by the following formula: 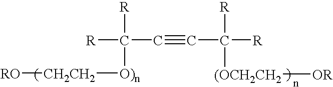
wherein: each n is independently a number between 2 and about 20 and are chosen such that the weight percent of polyoxyethylene in the surfactant is between 20 and 80 percent, preferably between 30 and 60 percent; and

each R is selected independently from one another as an alkyl or an aryl group that may be substituted or unsubstituted and that contain from 2 to about 20 carbon atoms whose skeletal chain may be straight-chained, branched, or, if sufficiently large, cyclic, or any combination thereof; such skeletal chain can also optionally include one or more catenary heteroatoms such as oxygen, hexavalent sulfur, and trivalent nitrogen atoms bonded to the carbon atoms of the skeletal chain.

Another class of useful nonionic polyoxyethylene-containing surfactants useful in the practice of the invention includes those organosiloxane compounds that may be represented generally by the following formula:

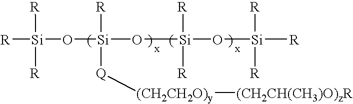

wherein: n, x, y, and z denote the number of repeating units in the depicted surfactant and are chosen such that the weight percent of polyethylene oxide in the surfactant is between 20 and 80 percent, preferably between 40 and 70 percent, and most preferably between 40 and 60 percent; It will be understood that the recurring siloxane units in the depicted formula may be randomly situated in the surfactant molecule;

Q is a multivalent, generally divalent, linking group, or is a covalent bond, that provides a means to link the silicon atom to the depicted oxyalkylene group; Q can comprise a heteroatom-containing group, e.g., a group containing —O—, —CO—, —CₙH₂ₙO—, or —OCₙH₂ₙO— where n is a number from 1 to 6; and

each R is selected independently from one another as an alkyl, alkoxy, aryl or aryloxy group that may be substituted or unsubstituted and that contain from 1 to about 20 carbon atoms whose skeletal chain may be straight-chained, branched, or, if sufficiently large, cyclic, or any combination thereof, the skeletal chain can also optionally include one or more catenary heteroatoms such as oxygen, hexavalent sulfur, and trivalent nitrogen atoms bonded to the carbon atoms of the skeletal chain. Useful silicone surfactants of the type depicted by the formula include ethoxylated polydimethylsiloxanes, such as Silwet™ L-77, commercially available from Union Carbide Corp.

Useful fluorochemical nonionic surfactants include fluoroaliphatic group-containing nonionic compounds that contain one or more blocks of water-solubilizing polyoxyalkylene groups in their structures. A class of such surfactants is described in U.S. Pat. No. 5,300,357 (Gardiner), whose descriptions are incorporated herein by reference. Generally, the fluorochemical surfactants useful in the invention include those represented below by Formula II.
(R_{f}-Q)ₙ-Z (II)
wherein:

R_{f} is a fluoroaliphatic group having at least 3, preferably at least 4, most preferably 4 to 7 fully-fluorinated carbon atoms that may be straight-chained, branched, or, if sufficiently large, cyclic, or any combination thereof. The skeletal chain in the fluoroaliphatic radical can include one or more catenary heteroatoms, such as oxygen, hexavalent sulfur, and trivalent nitrogen atoms bonded only to carbon atoms of the skeletal chain. Fully fluorinated fluoroaliphatic groups are preferred, but hydrogen or chlorine atoms may be present as substituents provided that not more than one atom of either if present for every two carbon atoms. While R_{f} can contain a large number of carbon atoms, compounds where R_{f} is not more than 20 carbon atoms will be adequate and preferred since larger radicals usually represent a less efficient utilization of the fluorine than is possible with shorter chains. Fluoroaliphatic radicals containing from about 4 to about 7 carbon atoms are most preferred. Generally, R_{f} will contain between about 40 and about 78 weight percent fluorine. The terminal portion of the R_{f} group preferably contains at least three fully fluorinated carbon atoms, e.g., C₃F₇—, and particularly preferred compounds are those in which the R_{f} group is fully or substantially completely fluorinated, as in the case where R_{f} is a perfluoroalkyl, e.g., CF₃(CF₂)ₙ—. Suitable Rf groups include, for example, C₄F₉—, C₆F₁₃CH₂CH₂—, and C₁₀F₂₁CH₂CH₂—.

Q in Formula II above is a multivalent, generally divalent, linking group, or is a covalent bond, that provides a means to link R_{f} with the depicted group Z, which is a nonionic, hydrophilic group; Q can comprise a heteroatom-containing group, e.g., a group such as —S—, —O—, —CO—, —SO₂—, —N(R)—, (where R is a hydrogen or a C₁ to C₆ substituted or unsubstituted alkyl group that may comprise a catenary heteroatom such as O, N, S), —CnH₂ₙ—(n=1 to 6); Q can comprise a combination of such groups such as would give, for example, —CON(R)CₙH₂ₙ—, —SO₂N(R)CₙH₂ₙ—, —SO₃C₆H4N(R)CₙH₂ₙ—, —SO₂N(R)CₙH₂ₙO[CH₂CH(CH₂Cl)O]_{g}CH₂CH(CH₂Cl)— (n=1 to 6; g 1 to 10), —SO₂N(CH₃)C₂H₄OCH₂CH(OH)CH₂—, —SO₂N(C₂H₅)C₂H₄OCH₂CH(OH)CH₂—, —SO₂N(H)CH₂CH(OH)CH₂NHC(CH₃)CH₂—, —(CH₂)₂S(CH₂)₂—, and —(CH₂)₄CH(CH₃)—;

Z in Formula II above is a nonionic, hydrophilic group comprising a poly(oxyalkylene) group, (OR′)ₓ, where R′ is an alkylene group having from 2 to about 4 carbon atoms, such as —CH₂CH₂—, —CH₂CH₂CH₂—, —CH(CH₃)CH₂—, and —CH(CH₃)CH(CH₃)—, and x is a number between about 4 and about 25; Z preferably contains a poly(oxyethylene) group. The oxyalkylene units in said poly(oxyalkylene) being the same, such as in poly(oxypropylene), or present as a mixture, such as in a heteric straight or branched chain of randomly distributed oxyethylene and oxypropylene units i.e., poly(oxyethylene-co-oxypropylene), or as in a straight or branched chain blocks of oxypropylene units. The poly(oxyalkylene) chain can be interrupted by or include one or more catenary linkages such as where Z includes a group of the formula —O—CH₂—CH(O)—CH₂—O—, providing such linkages do not substantially alter the water-solubilizing character of the poly(oxyalkylene) chain. The Z group may be terminated with a hydroxyl, alkyl ether (such as C₁ to C₂₀ alkyl ether), alkaryl ether, or fluoroalkyl ether, for example, —OCH₃, —OCH₂CH₃, —OC6H4C(CH₃)₂CH₂C(CH₃)₂CH₃, —OC₆ H₄(C₉H₁₉)₂, —OC₁₂H₂₅, —OC₁₄H₂₉, —OC₁₆ H₃₃, or —O—QR_{f} (where Q and R_{f} are as defined supra); and n is a number from 1 to 6.

Useful anionic surfactants include, but are not limited to, alkali metal and (alkyl)ammonium salts of: 1) alkyl sulfates and sulfonates such as sodium dodecyl sulfate and potassium dodecanesulfonate; 2) sulfates of polyethoxylated derivatives of straight or branched chain aliphatic alcohols and carboxylic acids; 3) alkylbenzene or alkylnaphthalene sulfonates and sulfates such as sodium laurylbenzene-sulfonate; 4) ethoxylated and polyethoxylated alkyl and aralkyl alcohol carboxylates; 5) glycinates such as alkyl sarcosinates and alkyl glycinates; 6) sulfosuccinates including dialkyl sulfosuccinates; 7) isothionate derivatives; 8) N-acyltaurine derivatives such as sodium N-methyl-N-oleyltaurate); 9) amine oxides including alkyl and alkylamidoalkyldialkylamine oxides; and 10) alkyl phosphate mono or di-esters such as ethoxylated dodecyl alcohol phosphate ester, sodium salt.

Representative commercial examples of suitable anionic sulfonate surfactants include, for example, sodium lauryl sulfate, available as TEXAPON™ L- 100 from Henkel Inc., Wilmington, Del., or as POLYSTEP™ B-3 from Stepan Chemical Co, Northfield, Ill.; sodium 25 lauryl ether sulfate, available as POLYSTEP™ B-12 from Stepan Chemical Co., Northfield, Ill.; ammonium lauryl sulfate, available as STANDAPOL™ A from Henkel Inc., Wilmington, Del.; and sodium dodecyl benzene sulfonate, available as SIPONATE™ DS-10 from Rhone-Poulenc, Inc., Cranberry, N.J., dialkyl sulfosuccinates, having the tradename AEROSOL™ OT, commercially available from Cytec Industries, West Paterson, N.J.; sodium methyl taurate (available under the trade designation NIKKOL™ CMT30 from Nikko Chemicals Co., Tokyo, Japan); secondary alkane sulfonates such as Hostapur™ SAS which is a Sodium (C ₁₄-C₁₇)secondary alkane sulfonates (alpha-olefin sulfonates) available from Clariant Corp., Charlotte, N.C.; methyl-2-sulfoalkyl esters such as sodium methyl-2-sulfo(C₁₂₋₁₆)ester and disodium 2-sulfo(C₁₂-C₁₆) fatty acid available from Stepan Company under the trade designation ALPHASTE™ PC-48; alkylsulfoacetates and alkylsulfosuccinates available as sodium laurylsulfoacetate (under the trade designation LANTHANOL™ LAL) and disodiumlaurethsulfosuccinate (STEPANMILD™ SL3), both from Stepan Company; alkylsulfates such as ammoniumlauryl sulfate commercially available under the trade designation STEPANOL™ AM from Stepan Company.

Representative commercial examples of suitable anionic phosphate surfactants include a mixture of mono-, di- and tri-(alkyltetraglycolether)-o-phosphoric acid esters generally referred to as trilaureth-4-phosphate commercially available under the trade designation HOSTAPHAT™ 340KL from Clariant Corp., as well as PPG-5 cetyl 10 phosphate available under the trade designation CRODAPHOS™ SG from Croda Inc., Parsipanny, N.J.

Representative commercial examples of suitable anionic amine oxide surfactants those commercially available under the trade designations AMMONYX™ LO, LMDO, and CO, which are lauryldimethylamine oxide, laurylamidopropyldimethylamine oxide, and cetyl amine oxide, all from Stepan Company.

Examples of useful amphoteric surfactants include alkyldimethyl amine oxides, alkylcarboxamidoalkylenedimethyl amine oxides, aminopropionates, sulfobetaines, alkyl betaines, alkylamidobetaines, dihydroxyethyl glycinates, imidazoline acetates, imidazoline propionates, ammonium carboxylate and ammonium sulfonate amphoterics and imidazoline sulfonates.

Representative commercial examples amphoteric surfactants include certain betaines such as cocobetaine and cocamidopropyl betaine (commercially available under the trade designations MACKAM™ CB-35 and MACKAM™ L from McIntyre Group Ltd., University Park, Ill.); monoacetates such as sodium lauroamphoacetate; diacetates such as disodium lauroamphoacetate; amino- and alkylamino-propionates such as lauraminopropionic acid (commercially available under the trade designations MACKAM 1L, MACKAM™ 2L, and MACKAM™ 151 L, respectively, from McIntyre Group Ltd.) and cocamidopropylhydroxysultaine (commercially available as MACKAM™ 50-SB from McIntyre Group Ltd.).

In addition, the adhesive layer may further contain a small amount of a solvent. The solvent may further aid as a solubilizing agent and carrier of the antimicrobial agent. It may also aid in transport of the antimicrobial compound through the polymer film as a carrier or by altering the polymer film itself, i.e. by decreasing the T_{g} of the polymer film.

The antimicrobial article may be prepared by combining the antimicrobial agent and the adhesive and coating the mixture onto the thermoplastic polymer layer. Any suitable coating method may be used. The antimicrobial agent is used in an amount sufficient to render the exposed surface (i.e. the surface opposite that coated with the adhesive layer) of the thermoplastic polymer layer antimicrobial upon migration of the antimicrobial agent.

The antimicrobial agent is typically used in an amount of at least about 0.25 wt. % based on the weight of the adhesive layer and more preferably in an amount of at least about 0.5 wt. %. The maximum amount of the antimicrobial agent is not critical; however, in case of an antimicrobial article consisting of only one layer of thermoplastic polymer, it is preferred to use the lowest amount possible so as not to impair the mechanical properties of the thermoplastic polymer layer. Generally, the amount of antimicrobial agent is between about 0.5 wt. % and 40 wt. %, and more preferably between about 1 wt. % and 30 wt. %. The actual concentration of the antimicrobial agent needed in the adhesive reservoir is highly dependent on the antimicrobial agent selected, the desired end use, and the duration of use. Some antimicrobial agents, such as antibiotics and silver compounds, can typically be used at much lower concentrations as they are inhibitory and efficacious at ppm levels.

The resultant antimicrobial article may be used, for example, for any use known for antimicrobial articles, but will typically have increased antimicrobial activity compared to the component thermoplastic polymers from which it is made. For example, a variety of medical and non-medical tapes may be prepared using the method of the invention. The tapes comprise a thermoplastic polymer backing; having an adhesive coated thereon, the adhesive containing an antimicrobial agent. The antimicrobial agent migrates from the adhesive layer to the backing layer (thermoplastic polymer layer) and other layers, rendering the article antimicrobial.

A variety of materials can be used to form the backing. The backing can be tearable or nontearable, elastic or inelastic, stretchable or nonstretchable, porous or nonporous. Backings can be in the form of single or multi-layer films, nonwoven films, porous films, foam-like films, and combinations of the foregoing (as previously described for the thermoplastic polymer layer). Backings can also be prepared from filled materials, such as, for example, filled films (e.g., calcium carbonate filled polyolefins).

Film backings can be made by any known method of film forming, such as, for example, extrusion, coextrusion, solvent casting, foaming, nonwoven technology, and the like. A backing can have a wide variety of thicknesses so long as it possesses sufficient integrity to be processable and with thicknesses preferably ranging from about 10 micrometers (i.e., microns) to about 250 micrometers.

Webs made of synthetic fibers or mixtures thereof can be used. Woven or nonwoven materials can be employed, with nonwoven materials being preferred for most applications. Melt-blown or spunbond techniques can be employed to make such nonwoven webs. Nonwoven webs can also be prepared on a Rando Webber (Rando Corporation, Macedon, N.Y.) air-laying machine or on a carding machine.

If the backing substrate is in the form of a laminate, additional components could be used, such as absorbent layers (e.g., gauze pads) for adhesive bandage products, or the like. If absorbent layers are used, they are typically thin, coherent, conformable, and able to flex and not interfere with the stretch removable characteristics of the articles, although they can be stretchable or not.

If a laminate, there may be one or more additional layers, which can be a breathable, liquid impervious film. Typically this film is the outermost (i.e., top) layer. Examples of film materials include polyurethanes, polyolefins, metallocene polyolefins, polyesters, polyamides, polyetheresters, and A-B-A block copolymers, such as KRATON copolymers available from Shell Chemical Co. Preferably, the outermost layer is a film that is substantially impervious to fluids, such as could arise from the external environment, yet permit passage of moisture vapor, such that the adhesive article is breathable (typically, having a moisture vapor transmission rate (MVTR) of at least about 500 g/m²/day).

The backing can optionally include fibers, which may be absorbent or nonabsorbent, and typically they are non-water absorptive. The fiber structures useful in the backing substrate of the present invention can include a multilayer configuration, a coated configuration, and a solid homogeneous configuration.

Representative examples of materials suitable for the backing of the adhesive article of this invention include polyolefins, such as polyethylene, including high density polyethylene, low density polyethylene, linear low density polyethylene, and linear ultra low density polyethylene, polypropylene, and polybutylenes; vinyl copolymers, such as polyvinyl chlorides, both plasticized and unplasticized, and polyvinyl acetates; olefinic copolymers, such as ethylene/methacrylate copolymers, ethylene/vinyl acetate copolymers, acrylonitrile-butadiene-styrene copolymers, and ethylene/propylene copolymers; acrylic polymers and copolymers; polycaprolactones; and combinations of the foregoing. Mixtures or blends of any plastic or plastic and elastomeric materials such as polypropylene/polyethylene, polyurethane/polyolefin, polyurethane/polycarbonate, polyurethane/polyester, can also be used.

The article of the invention may be applied to a wound dressing construction. A typical wound dressing includes a porous or non-porous facing layer (i.e. a wound-facing layer), having an adhesive layer comprising an antimicrobial agent, to provide a fluid permeable barrier between the wound site and an absorbent layer (such as an absorbent gel layer), and a backing layer. The antimicrobial agent migrates from the adhesive layer to the adjoining layers to render the wound dressing antimicrobial. Hence the antimicrobial adhesive prevents the growth of fungi and bacteria in the dressing. The wound dressing of this invention is particularly useful for wet dressings, i.e. dressings which retain a large amount of moisture and wound fluid (which normally provides an ideal environment for bacterial growth, but which growth is retarded in this construction).

The facing layer allows transport of moisture (i.e. fluid and vapor) from the wound to the gel layer and may isolate the wound from other components of the dressing. The facing layer is preferably soft, flexible, conformable, non-irritating and non-sensitizing. Any of a variety of polymers may be used including polyurethane, polyethylene, polypropylene, polyamide or polyester materials. Further, the facing layer may be in the form of moisture vapor permeable films, perforated films, woven-, non-woven or knit webs or scrims. A preferred facing layer comprises a polyurethane film. With reference to the instant invention, an embodiment of the thermoplastic polymer layer is the facing layer of a wound dressing.

In one useful embodiment, the facing layer is conformable to animal (including human) anatomical surfaces, has a moisture vapor transmission rate (MVTR) of at least 300 grams per square meter per 24 hours at 80% relative humidity differential at 40° C. (per method of U.S. Pat. No. 5,733,570 (Chen et al.)), is impermeable to liquid water throughout substantially its entire imperforate area and contains perforations for passing wound exudate through the facing layer. This means that the facing layer does not pass liquid water under normal wound treatment conditions except at the places in the facing layer that are positively perforated to allow the exudate to pass into the reservoir.

The preferred moisture vapor transmission rate of the facing layer is at least 600 grams per square meter per 24 hours at an 80% relative humidity differential at 40° C. The facing layer may further comprise a pressure sensitive adhesive layer. The adhesive coated facing layer preferably has the aforesaid MVTR. Therefore, if the facing layer is impermeable to liquid water except for the perforation means, the adhesive can be permeable to liquid water and vice versa. Porous or non-porous facing layers such as perforated polyamide, polyester, polypropylene, polyethylene, polyether-amide, polyurethanes, chlorinated polyethylene, styrene/butadiene block copolymers (KRATON brand thermoplastic rubber, Shell Chemical Company, Houston, TX) and poly(vinyl chloride) and those described in U.S. Pat. No. 3,121,021 (Copeland) that are covered with a pressure sensitive adhesive that is not permeable to liquid water can be used for the facing layer. Optionally these films can be perforated. Additional porous materials include woven and non-woven substrates.

It is preferred that the facing layer have the above mentioned moisture vapor or liquid permeability (1) so that maceration of the skin under the wound dressing does not occur; (2) so that moisture build-up under the facing layer does not cause the facing layer and, therefore, wound dressing to be lifted off the skin; and (3) to enhance proximation of the wound edges. Preferred facing layers are thin polymeric films optionally coated with pressure sensitive adhesive which, in combination, have the above characteristics.

The perforation means in the facing layer are holes or slits or other perforations that conduct the passage of liquid water or wound exudate from the wound into the absorbent layer of the wound dressing. The perforations may additionally extend through an adhesive layer, if the front surface of the facing film (that surface facing toward the wound) is coated with a pressure sensitive adhesive layer.

A backing layer may be present in all of the embodiments of a wound dressing. Preferably the backing layer is conformable to animal anatomical surfaces, impermeable to liquid water and has a moisture vapor transmission rate of at least 600 grams per square meter per 24 hours at an 80% relative humidity differential at 40° C. The backing layer, in combination with a facing layer, may be constructed to form a reservoir (e.g., a pouch or envelope) that surrounds the gel layer, into which the exudate from the wound passes. This reservoir does not permit liquid water or exudate to pass out of it. Instead, the gel layer absorbs the exudate, and moisture in the exudate passes through the backing layer in a vapor form into the atmosphere. The dressing permits wound exudate to be rapidly removed from the wound site and prevents liquids or bacteria from outside the dressing to contaminate the wound site. The antimicrobial agent in the adhesive layer of the wound dressing renders the article antimicrobial.

In order to remove moisture vapor, the moisture vapor transmission rate of the backing layer is at least as above noted, and preferably at least 1200 grams per square meter per 24 hours at an 80% relative humidity differential at 40° C.

The preferred embodiments for the facing and backing layers are thin conformable polymeric films. Generally the films are about 12 microns to about 50 microns in thickness, preferably about 12 microns to about 25 microns. Conformability is somewhat dependent on thickness, thus the thinner the film the more conformable the film. Reference has been made herein to the films utilized in the medical article (e.g., wound dressing) of the present invention being conformable to animal anatomical surfaces. This means that when the films of the present invention are applied to an animal anatomical surface, they conform to the surface even when the surface is moved. The preferred films are conformable to animal anatomical joints. When the joint is flexed and then returned to its unflexed position, the film stretches to accommodate the flexation of the joint but is resilient enough to continue to conform to the joint when the joint is returned to its unflexed condition.

Examples of films which are useful as facing or backing layers include polyurethanes such as those available under the trade designation ESTANE from B. F. Goodrich, Cleveland, Ohio, elastomeric polyester such as those available under the trade designation HYTREL from E. I. duPont deNemours & Co., Wilmington, Del., blends of polyurethanes and polyesters, polyvinyl chlorides, and polyether-amide block copolymers such as those available under the trade designation PEBAX available from Elf-Atochem. Particularly preferred films for use in the present invention are polyurethane and elastomeric polyester films. The polyurethane and elastomeric polyester films exhibit a resilient property that allows the films to have good conformability.

Particularly useful films include “spyrosorbent” films having a differential moisture vapor transmission rate (MVTR). Dressings incorporating spyrosorbent films not only manage wound exudate by absorption, but have the ability to adjust the moisture vapor transmission properties in response to the amount of exudate. Such spyrosorbent films are hydrophilic, moisture vapor permeable and have a relatively high MVTR (wet), and have a differential MVTR ratio (wet to dry) that is greater than one, and preferably greater than 3:1. The dry MVTR is greater than about 2600 g /m²/24 hrs, preferably about 3000 to 4000 g/m²/24 hrs. A particularly preferred spyrosorbent film, useful as a backing layer, is a segmented polyurethane such as a segmented polyether polyurethane urea based on polytetramethylene glycol and polyethylene glycol polyols. Such a spyrosorbent films are described in U.S. Pat. Nos. 5,653,699 and 4,849,458 (Reed et al.).

Another suitable backing layer is a fluid control film having at least one microstructures-bearing surface with channels that permit directional control of a liquid. This film can be used to transport a fluid to a remote site and thereby facilitate wicking away of a fluid (e.g., wound exudate). Such a film is disclosed in U.S. Pat. No. 6,420,622 (Johnston et al.).

Many different constructions of a wound dressing are possible with the facing layer, the gel layer, the backing layer and the adhesive layer (with the adhesive layer containing an antimicrobial agent). In one embodiment, the areas of the facing layer and the backing layer are greater than that of the gel layer and the facing layer is bonded to the backing layer, thereby forming a pouch, with the gel disposed between the two. In another embodiment, one of the facing or backing layers may be substantially the same area as the gel layer, and the other of greater area. The greater area of the facing or backing layer forms a periphery to which an adhesive layer and a release liner may be attached. It will further be understood that the facing and/or backing layer may be attached or bonded to the adjacent surface of the gel layer to form a contiguous layer construction, in which the backing and facing layers may be the same or of greater area than the gel layer. Alternatively, the backing and facing layers may be bonded to each other, and may or may not be bonded to the gel layer. In these last constructions, the gel layer is constrained within a pouch created by the attachment of the facing and backing layers to each other. The layers may be bonded to each other by any conventional means such as adhesives, heat-sealing, or other bonding means.

It is preferred that the facing and backing layers be at least translucent and more preferably sufficiently transparent so that the wound site to which they are applied can be viewed through the medical article. It is advantageous to view and evaluate the wound and healing thereof without removal of the wound dressing to avoid unnecessary handling of the wound site and exposure of the wound to the environment, which reduces the likelihood of contamination, and avoids the need to cleanse the wound as would be the case were the dressing to be removed. It is preferred that the dressing be both transparent and colorless so that the color of the wound, exudate, and periwound skin may also be evaluated. Preferred transparent films for use as facing and backing layers that allow visual inspection of the wound site include polyurethane films such as those available under the trade designation ESTANE from B. F. Goodrich, Cleveland, Ohio; elastomeric polyesters such as those available under the trade designation HYTREL from E. I. duPont deNemours & Co., Wilmington, Del.; and, polyether block amides such as those available under the trade designation PEBAX from Elf Atochem North America, Philadelphia, Pa. Other useful films are those describes in U.S. Pat. No. 4,499,896 (Heinecke); U.S. Pat. No. 4,598,004 (Heinecke); and U.S. Pat. No. 5,849,325 (Heinecke et al).

The wound dressing further comprises an adhesive layer (containing an antimicrobial) on all or part of the facing layer (i.e. thermoplastic film layer). The presence of the adhesive of the facing layer normally reduces the moisture vapor permeability of the facing layer. Therefore it is preferred that the facing layer is adhesive coated prior to adding a plurality of perforations to the facing layer. The wound exudate therefore can readily pass through a perforated adhesive coated facing layer. Preferably, both the facing and backing layers are precoated with an adhesive layer to both facilitate bonding of the backing layer to the facing layer (forming a pouch), and bonding of the facing film to the wound site.

The facing layer may be attached to the wound site by means of adhesive (containing an antimicrobial) that can be continuous or pattern coated. The preferred adhesive which can be used with the wound dressings of the present invention are the normal adhesives which are applied to the skin such as those described in U.S. Pat. No. Re. 24,906 (Ulrich). Other useful adhesives are those described in U.S. Pat. No.3,389,827 and acrylic adhesives such as iso-octyl acrylate IN-vinyl pyrrolidone copolymer adhesives and crosslinked acrylate adhesives such as for example those described in U.S. Pat. No. 4,112,213 (Waldman).

The adhesive may optionally be a microsphere adhesive with low trauma properties as described in U.S. Pat. No. 5,614,310 (Delgado et al.); a fibrous adhesive with low trauma properties as described in U.S. Pat. No. 6,171,985 B1 (Joseph et al.); or have especially good adhesion to wet skin, such as the adhesives described in U.S. Pat. No. 6,198,016 B 1 (Lucast et al.), and International Publication Nos. WO 99/13866 and WO 99/13865; multilayered adhesives as disclosed in U.S. Pat. Publication No. 2001/0051178 A1 (Blatchford et al.). A particularly preferred adhesive includes 15 wt-% acrylic acid, 15 wt-% methoxypolyethylene oxide 750 acrylate, 70 wt-% isooctyl acrylate, prepared according to Example I of U.S. Pat. No. 5,849,325 (Heinecke et al.).

The adhesive (containing an antimicrobial agent) may be chosen to be permeable to water or wound exudate, or the adhesive may be pattern coated on the front surface of the wound dressing (i.e. the surface in contact with the wound site, whether it is the front surface of the facing or backing layers) so as to not impede the flow of exudate to the gel layer, i.e. the adhesive may be coated at the periphery of the wound dressing. Alternatively, the adhesive layer may be perforated as described for the facing film to provide a fluid path for the exudate.

When pattern coated, such as at the periphery of a film layer in a wound dressing construction, the antimicrobial activity is not limited to the areas of film adjacent the patterned adhesive layer. Rather, it is believed that the antimicrobial agent will continue to migrate through the thermoplastic polymer layer to areas distal, rendering the entire surface of the thermoplastic film layer antimicrobial.

A release liner may be attached to the adhesive layer for ease of handling. Examples of release liners are liners made of or coated with polyethylene, polypropylene and fluorocarbons and silicone coated release papers or polyester films. Examples of the silicone coated release papers are POLYSLIK S-8004, 83 pound (135.4 g/m²) bleached silicone release paper supplied by H.P. Smith Co., Chicago, Ill., and 80 pound (130.5 g/m²) bleached two-sided silicone coated paper (2-80-BKG-1 57) supplied by Daubert Chemical Co., Dixon, Ill..

A wound dressing of the present invention may also include a frame that allows the dressing to be more easily applied to the wound. The frames are made of a relatively rigid material that maintains the shape of the dressing during handling and application to the wound site. The frame is generally releasably adhered to the back surface of the backing film and is removed after application of the wound dressing. Suitable frames are described in U.S. Pat. No. 5,531,855 (Heinecke et al.) and U.S. Pat. No. 5,738,642 (Heinecke et al.).

The antimicrobial articles are also useful as antimicrobial surfaces for use in food preparation and packaging, clean rooms, flooring, including carpeting, vapor barriers in building construction, shoe liners, protective films for display graphics and other such uses. The antimicrobial articles are also useful in the preparation, packaging and dispensing of pharmaceuticals or other medicaments.

In particular, the antimicrobial article may be used as a disposable surface for food preparation in commercial and residential kitchens. Such an article may be in the form of individual sheets, in a roll or in a set of stacked sheets. For example, a section of antimicrobial article may be unwound from a roll and secured to a substrate with the adhesive layer. In another embodiment, the invention provides a plurality of antimicrobial articles in the form of a stack, such as an (PA)n construction where P represents the thermoplastic polymer layer, A represents the adhesive layer, and n is greater than 1, e.g. 2 to 100. Individual articles may be removed from the stack and used as desired, or the stack per se may be secured to a substrate surface by means of the adhesive layer of the lowermost article. Fresh antimicrobial surfaces may be provided by removal of the uppermost article. In such a stack, the surface of the thermoplastic polymer layer may be treated with a release layer to allow subsequent sheets to be removed from the stack, or the construction may provide a release liner between adjacent articles. Alternatively, such articles may be provided with a removable or repositionable adhesive. Such articles may be used, then disposed of when contaminated; ensuring a clean antimicrobial surface.

It may be desirable in such an article to use a nonductile polymer for the thermoplastic polymer layer so that food may be cut, but the antimicrobial article resists cutting. Examples of such nonductile polymers include, but are not limited to, materials from the following classes: biaxially oriented polyethers; biaxially oriented polyesters; biaxially oriented polyamides; acrylic polymers such as poly(methyl methacrylate); polycarbonates; polyimides; cellulosics such as cellulose acetate, cellulose (acetate-co-butyrate), cellulose nitrate; polyesters such as poly(butylene terephthalate), poly(ethylene terephthalate); fluoropolymers such as poly(chlorofluoroethylene), poly(vinylidene fluoride); polyamides such as poly(caprolactam), poly(amino caproic acid), poly(hexamethylene diamine-coadipic acid), poly(amide-co-imide), and poly(ester-co-imide); polyetherketones; poly(etherimide); polyolefins such as poly(methylpentene); aliphatic and aromatic polyurethanes; poly(phenylene ether); poly(phenylene sulfide); atactic poly(styrene); cast syndiotactic polystyrene; polysulfone; silicone modified polymers (i.e., polymers that contain a small weight percent (less than 10 weight percent) of silicone) such as silicone polyamide and silicone polycarbonate; ionomeric ethylene copolymers such as poly(ethylene-co-methacrylic acid) with sodium or zinc ions, which are available under the trade designations SURLYN-8920 and SURLYN-9910 from E. I. duPont de Nemours, Wilmington, Del.; acid functional polyethylene copolymers such as poly(ethylene-co-acrylic acid) and poly(ethylene-co-methacrylic acid), poly(ethylene-co-maleic acid), and poly(ethylene-co-fumaric acid); fluorine modified polymers such as perfluoropoly(ethyleneterephthalate); and mixtures of the above polymers such as a polyimide and acrylic polymer blend, and a poly(methylmethacrylate) and fluoropolymer blend.

Such disposable articles may also comprise a removable or repositionable adhesive. A removable adhesive typically has a peel strength less than a conventional aggressively tacking PSA, for example a 180 degree peel strength (from a painted steel substrate employing a peel rate of 30.5 cm/min) of less than 8 N/cm, more particularly less than 6 N/cm. For purposes of this invention, an adhesive is considered to be “removable”, if after final application to an intended substrate the sheet material can be removed without damage to the substrate at the end of the intended life of the article at a rate in excess of 25 feet/hour (7.62 meters/hour) by hand with the optional use of heat. More preferably, the adhesive layer is a repositionable adhesive layer. For the purposes of this invention, “repositionable” refers to the ability to be, at least initially, repeatedly adhered to and removed from a substrate without substantial loss of adhesion capability. A repositionable adhesive usually has a peel strength, at least initially, to the substrate surface lower than that for a conventional aggressively tacky pressure sensitive adhesive.

Useful repositionable pressure sensitive adhesives include those described in U.S. Pat. No. 5,571,617 (Cooprider, et al.), entitled “Pressure Sensitive Adhesive Comprising Tacky Surface Active Microspheres”; or an adhesive from the class of adhesives based on solid inherently tacky, elastomeric microspheres, such as those disclosed in U.S. Pat. No. 3,691,140 (Silver), U.S. Pat. No. 3,857,731 (Merrill et al.), U.S. Pat. No. 4,166,152 (Baker et al.), although not limited to these examples.

The invention is further illustrated by means of the following examples without the intention to limit the invention thereto.

### EXAMPLES

These examples are merely for illustrative purposes only and are not meant to be limiting on the scope of the appended claims. All parts, percentages, ratios, etc. in the examples and the rest of the specification are by weight, unless noted otherwise. Solvents and other reagents used were obtained from Aldrich Chemical Company, Milwaukee, Wis. unless otherwise noted.

Test Methods

Surface Wetting Screening Test

This test is a qualitative measure of the surface wetting ability of a surface. A set volume of 10 microliters of deionized water was slowly deposited from a syringe directly onto the top surface of the material to be tested and observation was made whether the water droplet wets the surface or beads up during a period of about 15 minutes. The results are presented as “Wets” if the droplet wet the surface or “Beaded Up” if the droplet beaded up on the surface.

Zone of Inhibition Assay

This test is a semi-qualitative measure of the ability of a surface to inhibit microbial growth. It is performed by preparing separate solutions of Staphylococcus aureus (S. Aureus, American Type Culture Collection (ATCC) #25923), Escherichia coli (E. coli, ATCC #12229) and Candida albicans (C. albicans, ATCC #10231) at concentrations of approximately 1×10⁸ colony forming units (cfu) per milliliter (ml) in Phosphate Buffered Saline (PBS). These suspensions are used to prepare microbial lawns by dipping a sterile cotton applicator into the solution and swabbing the dry surface of separate trypticase soy agar (TSA) plates in three different directions. Three 7-millimeter disks from each sample are placed onto an inoculated plate and pressed firmly against the agar surface with sterile forceps to ensure complete contact. The plates are then incubated at 28° C.±1° C. for 24 hours. The area directly under and surrounding the samples is examined for microbial growth. Results of the inhibition assay are the average of three disks per sample. The zone of inhibition is reported as the diameter of the zone including the 7-mm sample disk. A primary zone (1°) shows no visible growth within it. A secondary zone (2°) shows inhibited growth within it. Some samples may have only one type of zone, while others may have both.

Bioluminescence Assay This test is a semi-qualitative measure of the ability of a surface to inhibit microbial growth. Bacteria with the “lux” gene inserted within through plasmid gene insertion are analyzed with a light intensity-measuring camera. As the antimicrobial takes effect, the luminosity of the sample decreases. The strain of bacteria used was E. coli DH5α ‘lux’ on LB Amp agar. E. coli is a gram-negative bacteria. Different concentrations of bacteria were used. These concentrations were based on optical density tests with a UV-Vis spectrometer, basing an absorbance of 1.0 (visible) on the presence of 10⁹ bacteria per mL. Through these tests the most visible results appeared with a concentration of 10⁹ bacteria. A 0.1 mL sample was added to each filter, which was then suctioned and placed on the agar plate. One uncovered control remained, while the other filters were covered with adhesive disks of both the negative controls and the antimicrobial mixtures. These disks were left on for varying amounts of time, generally periods of two, four, and six hours, after which they were removed. Readings were taken with the light intensity camera with an exposure time of 1 min., a discriminator level of 50, and a time interval of 1 hour.

Adhesion to Steel

A strip of test tape of 1 inch (2.5 cm) width was applied to a clean plate of #304 stainless steel with dimensions of 2 inches×5 inches× 1/16 inch (5×12.7×0.15 centimeters) having a bright annealed finish. The tape was rolled down with 2 passes of a 4.5 kilogram roller. Using a tensile tester, the tape was peeled at an angle of 180 degrees and at a speed of 12 inches/minute (300 millimeters/minute). The peel force was recorded in ounces/inch and converted to Newtons/2.5 centimeters. The average values were reported.

Table of Abbreviations

| Abbreviation | |
|---|---|
| or Trade | |
| Designation | Description |
| Adhesive-1 | A water-based latex adhesive prepared generally according to the |
| | procedure described in WO 01/81491 A1 (Loncar), Examples 6 and 7, by |
| | blending: 42.7 parts by weight of a dispersion of hollow tacky |
| | microspheres prepared as generally described in WO 92/13924 |
| | (Steelman, et al.), Example 1; 48.8 parts of an acrylate pressure-sensitive |
| | adhesive commercially available from 3M Company, St. Paul, MN, |
| | under the trade designation “FASTBOND 49”; 0.9 part by weight of an |
| | acrylic resin solution available from Rohm & Haas Company, |
| | Philadelphia, PA, under the trade designation “ACRYSOL ASE-60”; 2.5 |
| | parts by weight of n-octanol; 5 parts by weight of a mixture of 58 parts of |
| | water, 3 parts of lithium hydroxide monohydrate, and 39 parts of |
| | ammonium hydroxide; and 0.1 part by weight of a defoamer available |
| | under the trade designation “FOAMASTER JMY” from Cognis Corp., |
| | Ambler, PA. |
| Adhesive - 2 | Packaged acrylic wet stick adhesive prepared as described in U.S. Pat. |
| | No. 6,518,343 Polymerization Process B using the monomers 2- |
| | ethylhexyl acrylate/acrylic acid/PLURONIC 25R4 in the weight ratio |
| | 65/15/20. |
| Adhesive - 3 | Adhesive prepared as described in US Patent Publication Number |
| | 20030175503 Example 8 using the monomers 2-ethylhexyl |
| | acrylate/DMAEAMS/AM90G in the weight ratio 75/20/5. |
| Additive - 1 | Propylene glycol monocaprylate (Lot #024898) from Uniqema, New |
| | Castle, DE, a fatty acid monoester (FAME) registered with the EPA as |
| | an antimicrobial in 2003 by 3M, Co., St. Paul, MN. |
| Additive - 2 | Sodium dioctylsulfosuccinate C₈H₁₇OOCCH₂CH(SO₃Na)COOC₈H₁₇, |
| | “AEROSOL OT-100”, from Cytec Industries, West Patterson, NJ. |
| Additive - 3 | Triclosan (2,4,4′-trichloro-2′-hydroxydiphenyl ether) (Lot |
| | #K01.2/02/07/107) from Rita Corp., Forney, TX. |
| Additive - 4 | “BETADINE” solution (containing 10% povidone-iodine; equal to 1% |
| | available iodine) Topical Antiseptic Bactericide/Virucide from the |
| | Purdue Frederick Company, Norwalk, CT 06850-3590. It was |
| | determined that the solution retains approximately 10 percent of its |
| | weight after air drying for 1 day. |
| Additive - 5 | Sorbitan monolaurate “SPAN 20” from Uniqema, New Castle, DE. |
| Additive - 7 | Lauricidin (glycerol monolaurate) |
| Additive - 8 | Silver Nitrate, reagent grade |
| Liner-1 | PET release liner of with release agent on both sides, “SCOTCHPAK |
| | TPK 6752” available from 3M Company, St. Paul, MN. |
| Liner-2 | PET liner “HOSTAPHAN 4507” available from Mitsubishi Polyester |
| | Film Co., Tokyo, Japan. |
| PET | Poly(ethylene terephthalate) |
| Fabric-1 | Nonwoven flashspun High Density Polyethylene fabric, Product Number |
| | “TYVEK” 1042B, having a basis weight of 40.7 grams/square meter |
| | (g/m²), available from E.I. du Pont de Nemours and Company, |
| | Wilmington, DE. |
| Fabric-2 | A spunlaced nonwoven fabric prepared by hydroentangling an air-laid |
| | web consisting of 30 percent by weight of rayon fibers (1.5 denier × 3.8 |
| | cm long, trade designation “Type B649”, obtained from Lenzing Fiber |
| | Corporation, Lowland, Tennessee), 60 weight percent of polyester staple |
| | fibers (2.0 denier × 3.8 cm long, trade designation “Type T224”, obtained |
| | from KoSa B.V., Houston, TX), and 10 weight percent of PET/coPET |
| | sheath/core bicomponent fibers (2.0 denier × 3.8 cm long, trade |
| | designation “Celbond Type T254”, obtained from KoSa B.V.). |
| | A conventional hydraulic entangling system consisting of 6 |
| | manifolds/jets (3 above and 3 below) was used. The basic operating |
| | procedure is described in U.S. Pat. No. 5,389,202 (Everhart et al.), the |
| | disclosure of which is incorporated herein by reference. Each manifold |
| | had an orifice diameter of 120 microns. Orifices were positioned in a |
| | single row at spacing of about 16 orifices per linear centimeter of |
| | manifold. Manifold water pressure was successively ramped up to 127 |
| | kg/cm², which generated high energy fine columnar water jets. The air |
| | laid web was passed under the manifolds at a line speed of about 10 |
| | m/min, and then dried. Prior to hydroentangling, a carded web was first |
| | passed through an oven to melt the sheath component of the bicomponent |
| | fibers thereby providing a somewhat cohesive air-laid web. The |
| | nonwoven fabric had a basis weight of 85 g/m² and a thickness of 0.5 |
| | mm. |
| Fabric-3 | Polypropylene meltblown microfiber nonwoven fabric, prepared as |
| | described in Wente, Van A., “Superfine Thermoplastic Fibers” in |
| | Industrial Engineering Chemistry, Vol. 48, page 1342 et seq. (1956), or |
| | in Report No. 4364 of the Naval Research Laboratories, published May |
| | 25, 1954, entitled “Manufacture of Superfine Organic Fibers,” by Wente, |
| | V. A.; Boone, C. D.; and Fluharty, E. L., having a basis weight of 21.5 |
| | g/m² and an average effective fiber diameter (EFD) of 20 microns. The |
| | average EFD of the web was calculated using an air flow rate of 32 |
| | L/min according to the method described in Davies, C. N., “The |
| | Separation of Airborne Dust and Particles,” Institution of Mechanical |
| | Engineers, London, Proceedings 1B, 1952. |
| Membrane-1 | A sample of a polypropylene based microporous membrane prepared by |
| | the thermally induced phase separation technique (U.S. Pat. No. 4,539,256 - |
| | Shipman et al., U.S. Pat. No. 4,726,989; U.S. Pat. No. 5,120,594 - Mrozinski). |
| | Sample was 0.18 millimeters thick, 40% porosity, and with |
| | 0.8 micrometer pore size. |
| Film-1 | A 15 micrometer thick extruded film of ESTANE 58237 thermoplastic |
| | polyurethane, available from Noveon, Inc., Cleveland, OH. |
| Film-2 | A 40 micrometer thick extruded film of HYTREL 4056 thermoplastic |
| | polyester elastomer, available from DuPont Engineering Polymers, |
| | Wilmington, DE. |
| PLURONIC | Triblock copolymer with poly(propylene oxide) end blocks and |
| 25R4 | poly(ethylene oxide) midblock commercially available from BASF, |
| | Mount Olive, N.J. |
| DMAEAMS | Dimethylaminoethyl acrylate dimethyl sulfate quaternary salt (Ageflex |
| | FA1Q80DMS) 80% aqueous solution commercially available from Ciba |
| | Specialty Chemicals, Woodbridge, NJ. |
| AM90G | Methoxy(polyethylene oxide) acrylate of approximately 450 molecular |
| | weight commercially available from Shin-Nakamura Chemicals, |
| | Wakayama City, Japan. |

### Example 1

Part I: Preparation of Adhesive Sample

A mixture of Adhesive-1 and 10% by weight of Additive-1 was prepared and coated at a thickness of 6 mils with a doctor knife onto Liner-1, and allowed to dry at room temperature for three days to give a dry adhesive thickness of approximately 2.4 mils. The final concentration of Additive-1 in the dried adhesive was approximately 21.7 % by weight.

Part II: Preparation and Testing of Laminates

Two tapes of the adhesive sample prepared in Part I above were prepared by laminating adhesive samples to two samples of Fabric-1. The release liners were removed from each of these tapes and the adhesive sides of each tape was laminated to a glass slide to form a 3 layer laminate. One laminate was placed to age in an 85° C. oven, the second laminate was aged at room temperature. The sample laminates were tested daily for up to 27 days by the Surface Wetting Screening Test using the test method described above. The results are shown in Table 1.

### Comparative Example C1

Part I: Preparation of Adhesive Sample

Adhesive-1 with no additive was coated as described for Example 1, Part I above.

Part II: Preparation and Testing of Laminates

Two tapes of the adhesive sample prepared in Part I above were prepared by laminating adhesive samples to two samples of Fabric-1. The release liners were removed from each of these tapes and the adhesive sides of each tape was laminated to a glass slide to form a 3-layer laminate. One laminate was placed to age in an 85° C. oven, the second laminate was aged at room temperature. The sample laminates were tested daily for up to 27 days by the Surface Wetting Screening Test using the test method described above. The results are shown in Table 1.

### Example 2

Part I: Preparation of Adhesive Sample

Adhesive-1 with Additive-1 was coated as described for Example 1, Part I above.

Part II: Preparation and Testing of Laminates

Two tapes of the adhesive sample prepared in Part I above were prepared by laminating adhesive samples to three samples of Fabric-2. The release liners were removed from each of these tapes and the adhesive sides of each tape was laminated to a glass slide to form a 3 layer laminate. One laminate was placed to age in an 85° C. oven, the second laminate was aged at room temperature. The sample laminates were tested daily for up to 27 days by the Surface Wetting Screening Test using the test method described above. The results are shown in Table 1.

### Comparative Example C2

Part I: Preparation of Adhesive Sample

Adhesive-1 with no additive was coated as described for Example 1, Part I above.

Part II: Preparation and Testing of Laminates

Two tapes of the adhesive sample prepared in Part I above were prepared by laminating adhesive samples to three samples of Fabric-2. The release liners were removed from each of these tapes and the adhesive sides of each tape was laminated to a glass slide to form a 3 layer laminate. One laminate was placed to age in an 85° C. oven, the second laminate was aged at room temperature. The sample laminates were tested daily for up to 27 days by the Surface Wetting Screening Test using the test method described above. The results are shown in Table 1.

### Example 3

Part I: Preparation of Adhesive Sample

Adhesive-1 with Additive-1 was coated as described for Example 1, Part I above.

Part II: Preparation and Testing of Laminates

Two tapes of the adhesive sample prepared in Part I above were prepared by laminating adhesive samples to two samples of Fabric-3. The release liners were removed from each of these tapes and the adhesive sides of each tape was laminated to a glass slide to form a 3 layer laminate. One laminate was placed to age in an 85° C. oven, the second laminate was aged at room temperature. The sample laminates were tested daily for up to 27 days by the Surface Wetting Screening Test using the test method described above. The results are shown in Table 1.

### Comparative Example C3

Part I: Preparation of Adhesive Sample

Adhesive-1 with no additive was coated as described for Example 1, Part I above.

Part II: Preparation and Testing of Laminates

Two tapes of the adhesive sample prepared in Part I above were prepared by laminating adhesive samples to two samples of Fabric-3. The release liners were removed from each of these tapes and the adhesive sides of each tape was laminated to a glass slide to form a 3 layer laminate. One laminate was placed to age in an 85° C. oven, the second laminate was aged at room temperature. The sample laminates were tested daily for up to 27 days by the Surface Wetting Screening Test using the test method described above. The results are shown in Table 1.

### Example 4

Part I: Preparation of Adhesive Sample

A mixture of Adhesive-1 and 10% by weight of Additive-1 was prepared and coated at a thickness of 8 mils with a doctor knife onto Liner-1, and allowed to dry at room temperature for 1 day to give a dry adhesive thickness of approximately 4 mils. The final concentration of Additive-1 in the dried adhesive was approximately 21.7% by weight.

Part II: Preparation and Testing of Laminates

Three tapes of the adhesive sample prepared in Part I above were prepared by laminating adhesive samples to three samples of Membrane-1. The release liners were removed from two of these tapes and the adhesive sides of each tape was laminated to a glass slide to form a 3 layer laminate. One laminate was placed to age in an 80° C. oven, the second laminate was aged at room temperature. The sample laminates were tested daily for 3 days by the Surface Wetting Screening Test using the test method described above. The results are shown in Table 1.

The third laminate of the adhesive tape with Membrane-1 prepared above was allowed to age at room temperature for 21 days. The Membrane surface of the laminate was then carefully placed face down onto an inoculated agar surface and tested via the Zone of Inhibition Assay described above. The results are shown in Table 2.

### Comparative Example C4

Part I: Preparation of Adhesive Sample

Adhesive-1 with no additive was coated as described for Example 4, Part I above.

Part II: Preparation and Testing of Laminates

Three tapes of the adhesive sample prepared in Part I above were prepared by laminating adhesive samples to three samples of Membrane-1. The release liners were removed from two of these tapes and the adhesive sides of each tape was laminated to a glass slide to form a 3 layer laminate. One laminate was placed to age in an 80° C. oven, the second laminate was aged at room temperature. The sample laminates were tested daily for 3 days by the Surface Wetting Screening Test using the test method described above. The results are shown in Table 1.

The third laminate of the adhesive tape with Membrane-1 prepared above was allowed to age at room temperature for 15 days. The Membrane surface of the laminate was then carefully placed face down onto an inoculated agar surface and tested via the Zone of Inhibition Assay described above. The results are shown in Table 2.

### Example 5

Part I: Preparation of Adhesive Sample

A mixture of Adhesive-1, 5% by weight of Additive-2 and 5% by weight of Additive-3 was prepared and coated at a thickness of 8 mils with a doctor knife onto Liner-2, and allowed to dry at room temperature for 1 day to give a dry adhesive thickness of approximately 4 mils. The final concentrations of Additive-2 and Additive-3 in the dried adhesive were each approximately 10.8% by weight.

Part II: Preparation and Testing of Laminates

Three tapes of the adhesive sample prepared in Part I above were prepared by laminating adhesive samples to three samples of Membrane-1. One laminate was placed to age in an 80° C. oven, the second laminate was aged at room temperature. These sample laminates were tested after 1 day by the Surface Wetting Screening Test using the test method described above. The results are shown in Table 1.

The third laminate of the adhesive tape with Membrane- l prepared above was allowed to age at room temperature for 25 days. The Membrane surface of the laminate was then carefully placed face down onto an inoculated agar surface and tested via the Zone of Inhibition Assay described above. The results are shown in Table 2.

### Comparative Example C5

Part I: Preparation of Adhesive Sample

Adhesive-1 with no additive was coated as described for Example 5, Part I above.

Part II: Preparation and Testing of Laminates

Two tapes of the adhesive sample prepared in Part I above were prepared by laminating adhesive samples to two samples of Membrane-1. One laminate was placed to age in an 80° C. oven, the second laminate was aged at room temperature. The sample laminates were tested after 1 day by the Surface Wetting Screening Test using the test method described above. The results are shown in Table 1.

### Example 6

Part I: Preparation of Adhesive Sample

A mixture of Adhesive-1, 10% by weight of Additive-2 and 10% by weight of Additive-4 was prepared and coated at a thickness of 8 mils with a doctor knife onto Liner-2, and allowed to dry at room temperature for 1 day to give a dry adhesive thickness of approximately 4 mils. The final concentrations of Additive-2 and residual Additive-4 in the dried adhesive were approximately 23.3% and 2.3% by weight respectively.

Part II: Preparation and Testing of Laminates

Two tapes of the adhesive sample prepared in Example 6, Part I above were prepared by laminating adhesive samples to two samples of Membrane-1. One laminate was aged at room temperature and tested daily for 3 days by the Surface Wetting Screening Test using the test method described above. The result is shown in Table 1.

The second laminate of the adhesive tape with Membrane-1 prepared above was also allowed to age at room temperature for 4 days. The Membrane surface of the laminate was then carefully placed face down onto an inoculated agar surface and tested via the Zone of Inhibition Assay described above. The results are shown in Table 2.

### Comparative Example C6

Part I: Preparation of Adhesive Sample

Adhesive-1 with no additive was coated as described for Example 6, Part I above.

Part II: Preparation and Testing of Laminates

A tape of the adhesive sample prepared in Part I above was prepared by laminating adhesive sample to a sample of Membrane-1. The laminate was aged at room temperature. The sample laminate were tested daily for 3 days by the Surface Wetting Screening Test using the test method described above. The results are shown in Table 1.

Part II: Preparation and Testing of Laminates

Two tapes of the adhesive sample prepared in Part I above were prepared by laminating adhesive samples to two samples of Membrane-1. One laminate was placed to age in an 80° C. oven, the second laminate was aged at room temperature. The sample laminates were tested daily for 3 days by the Surface Wetting Screening Test using the test method described above. The results are shown in Table 1.

### Comparative Example C8

Part I: Preparation of Adhesive Sample

Adhesive-1 with no additive was coated as described for Example 7, Part I above.

Part II: Preparation and Testing of Laminates

A tape of the adhesive sample prepared in Part I above was prepared by laminating the adhesive sample to a sample of Film-1. The laminate was aged at room temperature for 8 days. The Film surface of the laminate was then carefully placed face down onto an inoculated agar surface and tested via the Zone of Inhibition Assay described above. The results are shown in Table 2.

### Example 8

Part I: Preparation of Adhesive Sample

An adhesive sample containing Adhesive-1, Additive-2, and Additive-3 was prepared in the same manner as described in Example 5, Part I above.

Part II: Preparation and Testing of Laminates

A tape of the adhesive sample prepared in Example 8, Part I above was prepared by laminating the adhesive sample to a sample of Film-1. The laminate was aged at room temperature. The laminate was aged at room temperature for 8 days. The Film surface of the laminate was then carefully placed face down onto an inoculated agar surface and tested via the Zone of Inhibition Assay described above. The results are shown in Table 2.

### Example 9

Part I: Preparation of Adhesive Sample

An adhesive sample containing Adhesive-1, Additive-2, and Additive-3 was prepared in the same manner as described in Example 5, Part I above.

Part II: Preparation and Testing of Laminates

A tape of the adhesive sample prepared in Example 9, Part I above was prepared by laminating the adhesive sample to a sample of Film-2. The laminate was aged at room temperature. The laminate was aged at room temperature for 8 days. The Film surface of the laminate was then carefully placed face down onto an inoculated agar surface and tested via the Zone of Inhibition Assay described above. The results are shown in Table 2.

### Comparative Example C9

Part I: Preparation of Adhesive Sample

Adhesive-1 with no additive was coated as described for Example 5, Part I above.

Part II: Preparation and Testing of Laminates

A tape of the adhesive sample prepared in Part I above was prepared by laminating the adhesive sample to a sample of Film-2. The laminate was aged at room temperature for 8 days. The Film surface of the laminate was then carefully placed face down onto an inoculated agar surface and tested via the Zone of Inhibition Assay described above. The results are shown in Table 2.

TABLE 1

| | Aging | | |
|---|---|---|---|
| Example | Temperature | Aging Time | Surface Wetting |
| 1 | Room Temperature | 6 | Wets |
| 1 | 85° C. | 20 | Beaded Up |
| C1 | Room Temperature | 27 | Beaded Up |
| C1 | 85° C. | 20 | Beaded Up |
| 2 | Room Temperature | 27 | Wets |
| 2 | 85° C. | 2 | Wets |
| C2 | Room Temperature | 27 | Wets |
| C2 | 85° C. | 20 | Beaded Up |
| 3 | Room Temperature | 6-9 | Wets |
| 3 | 85° C. | 20 | Beaded Up |
| C3 | Room Temperature | 27 | Beaded Up |
| C3 | 85° C. | 20 | Beaded Up |
| 4 | Room Temperature | 3 | Wets |
| 4 | 80° C. | 3 | Beaded Up |
| C4 | Room Temperature | 3 | Beaded Up |
| C4 | 80° C. | 3 | Beaded Up |
| 5 | Room Temperature | 1 | Wets |
| 5 | 80° C. | 1 | Wets |
| C5 | Room Temperature | 1 | Beaded Up |
| C5 | 80° C. | 1 | Beaded Up |
| 6 | Room Temperature | 3 | Wets |
| C6 | Room Temperature | 3 | Beaded Up |

TABLE 2

| | | Zone of | Biological Activity |
|---|---|---|---|
| | | inhibition | directly under |
| Example | Test Organism | (mm) | sample |
| 4 | S. aureus | None | No inhibition |
| | E. coli | None | No inhibition |
| | C. albicans | None | No growth |
| C4 | S. aureus | None | No inhibition |
| | E. coli | None | No inhibition |
| | C. albicans | None | No inhibition |
| 5 | S. aureus | 33 (1° Zone) | No growth |
| | | 40 (2° Zone) | |
| | E. coli | 22 (1° Zone) | No growth |
| | | 24 (2° Zone) | |
| | C. albicans | 8 (2° Zone only) | No growth |
| 6 | S. aureus | 9 (1° Zone only) | No growth |
| | E. coli | None | No inhibition |
| | C. albicans | None | No growth |
| C8 | S. aureus | None | No inhibition |
| | E. coli | None | No inhibition |
| | C. albicans | None | No inhibition |
| 8 | S. aureus | 36 (1° Zone) | No growth |
| | | 44 (2° Zone) | |
| | E. coli | 24 (1° Zone) | No growth |
| | | 25 (2° Zone) | |
| | C. albicans | 8 (2° Zone only) | No growth |
| 9 | S. aureus | 25 (1° Zone) | No growth |
| | | 31 (2° Zone) | |
| | E. coli | 14 (1° Zone) | No growth |
| | | 16 (2° Zone) | |
| | C. albicans | None | No inhibition |
| C9 | S. aureus | None | No inhibition |
| | E. coli | None | No inhibition |
| | C. albicans | None | No inhibition |

### Example 10

Part I: Preparation of Adhesive Sample

A mixture of Adhesive-2 (dissolved in ethyl acetate to give a 40% solution) and 5% by weight of Additive-7 was prepared and coated with a doctor knife onto Liner-1, and dried in a circulating-air oven at 93° C. (200° F.) for 15 minutes to give a dry adhesive thickness of approximately 30 micrometers (1.2 mil).

Part II: Preparation and Testing of Laminates

A tape of the adhesive sample prepared in Part I above were prepared by laminating adhesive samples to a sample of Film 1. The release liners were removed from the tape and the adhesion to steel was tested as described above. The results are shown in Table 3. The Bioluminescence testing for the tape was run as described above by placing a disk of this tape on the filter described in the test method. The results are shown in Table 3.

### Comparative Example C10

Part I: Preparation of Adhesive Sample

Adhesive-2 with no additive was coated as described for Example 10, Part I above.

Part II: Preparation and Testing of Laminates

The same procedure was followed as described in Example 10, Part II above. The results are shown in Table 3.

TABLE 3

| | Adhesion to Steel | |
|---|---|---|
| Example | (Newtons/2.5 cm) | Bioluminescence |
| 10 | 10.59 | Near-total kill at 10\hat{ }8 bacteria |
| | | Substantial kill at10\hat{ }9 bacteria |
| C10 | 3.71 | No kill observed |

### Example 11

Part I: Preparation of Adhesive Sample

Mixtures of Adhesive-3 and 2% by weight (Example 11 A) and 0.2% by weight (Example 11B) of Additive-8 were prepared and coated with a doctor knife onto Liner-1, and dried in a circulating-air oven at 93° C. (200° F.) for 15 minutes to give a dry adhesive thickness of approximately 30 micrometers (1.2 mil).

Part II: Pre p2aration and Testing of Laminates

A tape of the adhesive sample prepared in Part I above were prepared by laminating adhesive samples to a sample of Film-1. The Film surface of the tape was then carefully placed face down onto an inoculated agar surface and tested via the Zone of Inhibition Assay described above (using e. coli). The results are shown in Table 4.

### Comparative Example C11

Part I: Preparation of Adhesive Sample

Adhesive-3 with no additive was coated as described for Example 11, Part I above.

Part II: Preparation and Testing of Laminates

The same procedure was followed as described in Example 11, Part II above. The results are shown in Table 4.

TABLE 4

| Example | Zone of inhibition |
|---|---|
| 11A | 3 mm |
| 11B | 1 mm beyond disk |
| C11 | 0 mm |

1. An antimicrobial article comprising:
a thermoplastic polymer layer having a first surface and a second surface having an adhesive layer bonded to said second surface, said adhesive layer comprising an antimicrobial agent that migrates to said first surface of said polymeric layer.

2. The antimicrobial article of claim 1 wherein said polymeric layer comprises films, porous membranes, microporous membranes, and fibrous polymer layers.

3. The antimicrobial article of claim 1 wherein said antimicrobial agent is selected from iodine and iodophors, chlorhexidine salts; parachlorometaxylenol; triclosan; hexachlorophene; fatty acid monoesters of glycerol and propylene glycols; phenols; polyquaternary amines; quaternary silanes; hydrogen peroxide; silver and silver salts, silver oxide and silver sulfadiazine.

4. The antimicrobial article of claim 3 wherein said fatty acid monoesters are selected from glycerol monolaurate, glycerol monocaprylate, glycerol monocaprate, propylene glycol monolaurate, propylene glycol monocaprylate, propylene glycol monocaproate

5. The antimicrobial article of claim I wherein said adhesive layer provides a reservoir for gradual release of said antimicrobial agent.

6. The antimicrobial article of claim 5 wherein said adhesive layer comprises at least 0.25 wt. % of said antimicrobial agent.

7. The antimicrobial article of claim 6 wherein said adhesive layer comprises 0.25 to 40 wt. % of said antimicrobial agent.

8. The antimicrobial article of claim 1 wherein said polymeric layer is selected from polyesters, polyurethanes, polyamides and polyolefins.

9. The antimicrobial article of claim 1 wherein said polymeric layer is selected from homo- and copolymers of aliphatic mono-alpha olefins.

10. The antimicrobial article of claim 1 wherein said polymeric layer is selected from homo-, co- and terpolymers of ethylene and propylene.

11. The antimicrobial article of claim 1, wherein said adhesive layer is a pressure sensitive adhesive layer.

12. The antimicrobial article of claim 1 further comprising a release liner.

13. The antimicrobial article of claim 1, wherein said thermoplastic polymer layer is patterned.

14. The antimicrobial article of claim 1 wherein said adhesive layer is patterned.

15. The article of claim 1 wherein said thermoplastic polymer layer is a nonductile polymer layer.

16. The article of claims I wherein said adhesive layer is a repositionable adhesive layer.

17. The article of claim 1, wherein said thermoplastic polymer layer has a diffusion constant of greater than 10×10⁻¹⁰ cm²/s at 25° C.

18. The article of claim 1, wherein said thermoplastic polymer layer has a diffusion constant of greater than 100×10 cm²/s at 25° C.

19. The article of claim 1, wherein said adhesive layer further comprises a surfactant dispersed in said adhesive layer.

20. The article of claim 19, wherein said surfactant is selected form nonionic, amphoteric and anionic surfactants.

21. The article of claim 19 wherein said surfactant is present in amount of at least 0.05 wt. %, relative to the weight of the adhesive layer.

22. A multilayer article comprising a plurality of antimicrobial articles of claim 1.

23. The multilayer article of claim 22 in the form of a stack.

24. The article of claim 1, wherein said antimicrobial agent dispersed in said adhesive comprises an delivery system to facilitate the migration of such antimicrobial agents from the adhesive layer into adjoining thermoplastic polymer layer, and provide for replenishment of the antimicrobial agent.

25. The article of claim 1, wherein said thermoplastic polymer layer has a T_{g} of below about 0° C.

26. A method for providing an antimicrobial article comprising a thermoplastic polymer layer and an adhesive layer, comprising the steps of: (a) dispersing into an adhesive layer at least one antimicrobial agent; and (b) adhering the adhesive layer to a thermoplastic polymer layer, wherein the adhesive layer provides a antimicrobial agent reservoir for the polymer layer.

27. The method of claim 26 wherein said thermoplastic polymer layer comprises a film, a membrane, or a fibrous polymer layer.

28. The method of claim 26 wherein said antimicrobial agent is present in an amount sufficient to render said thermoplastic polymer layer antimicrobial.

29. The method of claim 26 wherein said adhesive layer comprises at least 0.25 wt. % of said antimicrobial agent.

30. The method of claim 26, wherein said thermoplastic polymer layer has a permeability coefficient of greater than 10×10⁻¹⁰ cm²/s at 25° C.

31. The method of claim 26 wherein said adhesive layer is coated onto said thermoplastic polymer layer.

32. The method of claim 26 wherein said thermoplastic polymer layer and said adhesive layer are coextruded.

33. The method of claim 26 wherein said polymeric layer is selected from polyesters, polyurethanes, polyamides and polyolefins.

34. The method of claim 26 wherein said polymeric layer is selected from homo-, co-and terpolymers of aliphatic mono- alpha olefins.

35. The method of claim 26 wherein said polymeric layer is selected from homo-, co-and terpolymers of ethylene and propylene.

36. The method of claim 26, wherein said adhesive layer is a pressure sensitive adhesive layer.

37. The method of claim 26, wherein said adhesive layer is a repositionable adhesive layer.

38. A wound dressing comprising the antimicrobial article of claim 1.

39. The wound dressing of claim 38 comprising a thermoplastic polymer facing layer, an adhesive layer on at least a portion of said facing layer, a backing layer, and a gel layer disposed between said facing and backing layers, said adhesive layer containing an antimicrobial agent.

40. A food preparation surface comprising the antimicrobial article of claim 1.
